(19) United States
(12) Patent Application Publication
(10) Pub. No.: US 2012/0330341 A1
(43) Pub. Date: Dec. 27, 2012

(54) Folded-Flat Aneurysm Embolization Devices

(76) Inventors: Frank P. Becking, Palo Alto, CA (US); Siddharth Loganathan, Sunnyvale, CA (US); Chi Vu, Milpitas, CA (US); Karl S. Halden, San Carlos, CA (US); Arturo S. Rosqueta, San Jose, CA (US)

(21) Appl. No.: 13/166,136

(22) Filed: Jun. 22, 2011

(51) Int. Cl. A61M 29/00; B23P 11/00

(52) U.S. Cl. 606/191; 29/428

(57) ABSTRACT

Embolic implants, methods of manufacture and delivery are disclosed. The subject implants are especially suitable for use is stent-caged aneurysm treatment.

### RELATED APPLICATIONS

The present filing claims the benefit of each of U.S. Patent Application Ser. No. 61/046,594 filed Apr. 21, 2008, Ser. No. 61/083,961 filed Jul. 28, 2008, Ser. No. 61/145,097 filed Jan. 15, 2009, and Ser. No. 12/465,475 filed May 13, 2009, each of which is incorporated herein by reference in its entirety, along with all references cited therein.

### BACKGROUND

Numerous companies have pursued ball type embolization devices for aneurysm treatment. Many of these, including embodiments in the above-referenced parent applications of the present application, are designed to be sized to substantially fill the sac of a given aneurysm. US Patent Application No. 2009/0025820 (Adams) discloses the use of a “string of pearls” type approach to filling aneurysms. The implant comprises a braid structure formed into multiple expandable ball segments separated by articulation segments. Each of Pub. No. 2007/0265656 (Amplatz, et al.), see, e.g., FIG. 15, and Pub. No. 2009/0112251 (Quain, et al.) disclose a similar approach. An approach described in U.S. Pat. Nos. 5,749,891 and 6,033,423 (Ken, et al.) is distinguished in the '820 Adams patent publication. These Ken et al. patents teach producing ball-shaped devices (e.g., generally oval or spherical) from a coil wound upon itself into a secondary shape (i.e., forming a “coil of a coil”) for filling aneurysms.

None of these references focus on use of a stent across the neck of the aneurysm to maintain embolization device position during treatment. However, the use of stents to “cage” an aneurysm for implant retention is well known. The commercially marketed NEUROFORM and ENTERPRISE stents are used in stent-assisted coiling. See also: U.S. Pat. No. 6,190,402 (Horton, et al.); U.S. Pat. Nos. 6,096,034; 6,168,592 and 6,344,041 (Kupiecki, et al.); U.S. Pat. No. 7,303,571 (Makower, et al.); U.S. Pat. No. 7,211,109 (Thompson) and U.S. Patent Publication No. 2008/0045996 (Makower, et al.).

Now-abandoned US Publication No. 2005/0060017 (Fischell, et al.) teaches a method of filling a stent-caged aneurysm with a plurality of expandable spherical or cylindrical filler bodies.

The present inventions are directed at a new type of implant and methods for using that implant with the advantages described and implied herein in embolizing stent-caged aneurysms.

### SUMMARY

Generally, braid-balls for stent-caged aneurysm embolization are described. More specifically, variations of the “folded-flat” implant architecture in the above-referenced parent application(s) are described and elaborated upon. In this regard, the implant variation presented in FIG. 9 of U.S. patent application Ser. No. 12/465,475 and PCT/US2009/041313 (Becking, et al.), reproduced herein at FIG. 2A, is of particular use. This implant employs the folded-flat architecture without hub securing ends of the implant braid opposite the folded-flat section. Among other reasons, such an implant is clearly distinguishable from the implants disclosed in US Pub. No. 2009/0082803 (Adams, et al). These implants all have free ends of braid at both ends of the device. The “folded-flat” architecture described herein specifically does not.

The folded-over and flattened braid of the subject implant imparts stability to the shaped body and provides a consistent closed end. The closure can be supplemented with a tie, or can rely only on the heatset determining its at-rest configuration. In either case, the closed end of the implant (positioned either proximally or distally in use) is one feature that enables the implant to reliably open after exiting a delivery catheter/microcatheter.

The shape of the implant may be substantially spherical. Otherwise, it may include a flat section adjacent the closed end with associated benefits as taught in U.S. patent application Ser. No. 12/942,209 (Becking, et al.), also incorporated herein by reference.

Alternatively (or additionally), the implant may be heatset over an ovaloid form. For a given size implant, such an approach decreases the radius around the waist of the implant. When compressed for delivery, the result is more stored energy to drive full shape recovery. This measure may be useful in overcoming any entangled/disordered free ends of the braid wire otherwise apt to interfere with full recovery of the implant shape from compression.

In the implant configurations described (i.e., folded-flat designs without an opposite-side hub) the wires are not subject to high stresses generated in compressing the braid for delivery. As such, even at larger wire diameters (i.e., in this context being upwards of 0.001″ and possibly at 0.002″ or more—depending on implant configuration) no superelasticity is required of the material. Nitinol may still be used to construct the implant given its convenience for heat setting and biocompatibility. However, other materials such as beta-Titanium are feasibly employed without loss of performance. Indeed, improved radiopacity may result in using a beta-Titanium alloy such as Ti-15Mo, Ti 11.5Mo-6Zr-4.5Sn or Ti-3Al-8V-6Cr-4Zr-4Mo.

The subject implants may include or omit radiopaque markers. Advantageous marker approaches are disclosed. Otherwise, the density of the braid from which the implant is constructed may be relied upon for radiopacity.

To achieve commercial success, low cost is preferred feature of the device. Simply put, with the number of implants to be used in the subject “multi-ball” treatment approach, per-piece production should be economical. By producing an implant without a hub and/or marker(s), cost is controlled. Additional savings can be realized by using the implant without a detachment system. Basic deployment with a low-cost pusher is a feasible when filling a stent (or neck-bridge) caged aneurysm.

Notably, the implant may be delivered with the folded section oriented proximally or distally. Generally, a proximal-facing fold will be preferred to help ensure that portions of the implant interacting with the caging stent remain patent. So-orienting the implant can avoid disruption of the open-end braid matrix from contact with stent struts. However, the implant can be inverted for use and such disruption avoided by selectively applying a polymer coating to maintain a consolidated relationship of the wire ends in the braid.

Coordinated use of the subject implant(s) is covered. Specifically, more than one may be packaged in a delivery sheath (or tube), a loading sheath, or a transfer sheath for use. The implants may be advantageously packaged in multiples of 2, 3, or more. Thus, a physician can select from a desired panel or pallet of options in determining how many implants are to be delivered in one “shot” or “go” at deployment.

Irrespective of such considerations, the subject implants offer profound performance benefit potential as compared to the current standard of care (comparable, in this case, to stent-assisted coiling). Various studies by the applicant have demonstrated that the braid matrix of the device is particularly effective in disrupting blood flow to embolize a site. Moreover, as compared to coils, the braid matrix provides for superior tissue colonization and growth to seal-off aneurysms.

The braid matrix is particularly effective as its density increases. For a given catheter crossing profile, a certain maximum braid configuration is possible. For example, folded-flat implants, intended to track to the neurovasculature through commercially available 3Fr/0.027 inch catheters (such as the commercially available REBAR or MARKSMAN), may be constructed from a 72×0.001″ braid configuration (as originally provided or etched thereto) or 96×0.0009″ braid configuration.

At these braid densities, implants produced with binary NiTi are sufficiently visible in the 4-6 mm diameter size range for individual viewing during intracranial use. They appear as dark cylindrical objects during catheter tracking and as a lighter circular or oval “halo” upon deployment. Still, other braid configurations may be employed in constructing the subject implants.

Regardless, implants in this size range may be preferred for intracranial aneurysm treatment as described. However, larger implant sizes may be employed as well. Even if not individually viewable without marker features, they can be visualized in aggregate. When delivered caged behind a stent (having cells sized to retain the expanded implants), escape is not a concern.

Naturally, implant sizing may vary. However, the sizing need not vary so significantly as with implants intended to fill a range of aneurysms alone. Rather in a multi-ball application, small, medium and large sizes will suffice for intracranial aneurysm treatment. The small size may have a volume equivalent to about a 4 mm sphere, a medium size of about that of a 5.5 mm sphere, and the large of about a 7 mm sphere—whether the devices are spherical, ovaloid or have other shapes.

Larger sizes in the given braid configurations are not as desirable for intracranial use. Not only is radiopacity decreased (i.e., at least in connection with 0.027 inch catheter-compatible braid configurations), but stability of the unconnected ends of braid degrades. For other indications/approaches, however, larger sized implants delivered through larger catheters may be desired. Indeed, for back-filling an aortic aneurysm around a stent, stent-grafts or flow disruptor to prevent endoleaks, balls as large as 1 cm or more may be beneficially used. Use of a larger catheters than suitable for neuro applications makes employing 144-end and 192-end braid configurations feasible in constructing the subject implants.

Aspects of the present invention include the subject implants and devices, kits in which they are included, methods of use and methods of manufacture. A number of aspects of such manufacture are discussed above. More detailed discussion is presented in connection with the figures below.

Other systems, methods, features and advantages will be or will become apparent to one with skill in the art upon examination of the following figures and detailed description. It is intended that all such additional systems, methods, features and advantages be included within this description, be within the scope of the inventive subject matter, and be protected by the accompanying claims. It is also intended that the inventive subject matter not be limited to the details of the example embodiments nor any representations made in this summary section.

### BRIEF DESCRIPTION OF THE FIGURES

The figures provided herein are not necessarily drawn to scale, with some components and features exaggerated for clarity. Variations of the inventive subject matter from the embodiments pictured are contemplated. Accordingly, the figures are not intended to limit the scope of the claims.

FIG. 1 shows an overview of the subject implant;

FIGS. 2A and 2B are side-sectional views of different implants (with and without a detachment system, respectively) suitable for stent-caged aneurysm treatment;

FIGS. 3A-3C show the implants in use to define a treatment system;

FIGS. 4A-4C are side-sectional views of different implant configurations;

FIGS. 5A-5D show different implant loading strategies;

FIGS. 6A and 6B illustrate a technique for presetting the shape of the implant fold;

FIGS. 7A and 7B illustrate another technique for the same; and

FIGS. 8A-8H are views illustrating stages of overall implant manufacture and an optional packaging approach.

### DETAILED DESCRIPTION

Various exemplary embodiments of the inventive subject matter are described below. Reference is made to these examples in a non-limiting sense. They are provided to illustrate more broadly applicable aspects of the inventive subject matter. Various changes may be made and equivalents may be substituted without departing from the true spirit and scope of the inventive subject matter. In addition, many modifications may be made to adapt a particular situation, material, composition of matter, process, process act(s) or step(s) to the objective(s), spirit or scope of the inventive subject matter. All such modifications are intended to be within the scope of the claims made herein.

### Implant System and Treatment Options

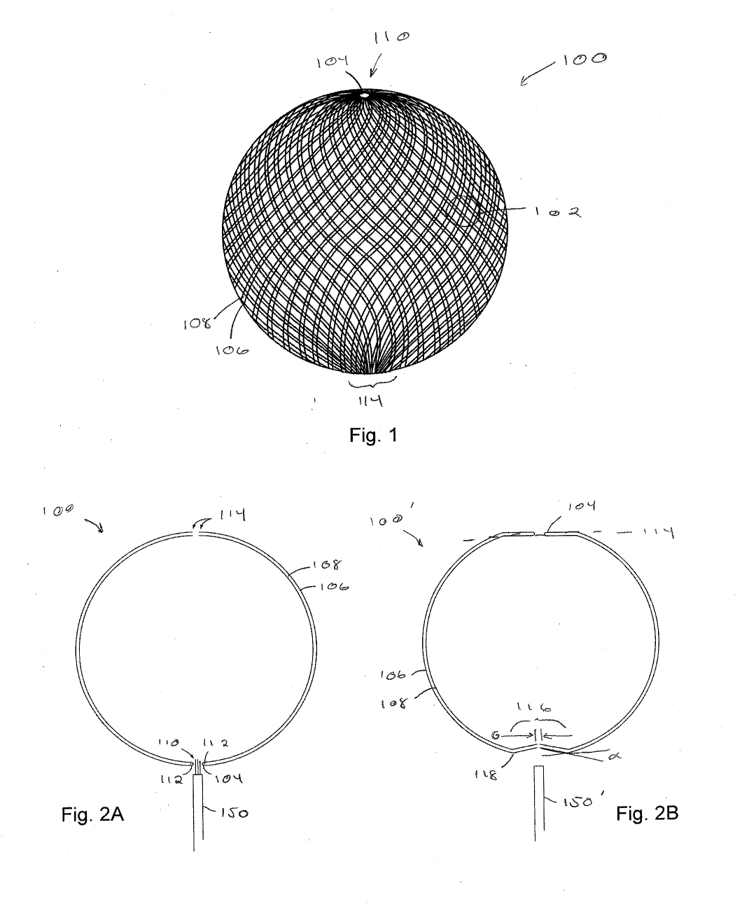

FIG. 1 shows an overview of the subject implant 100. It is formed from tubular braid stock 102 comprising a resilient material such as Nitinol that defines an open volume (generally round, spherical, ovular/ovoid, and the like) in an uncompressed/unconstrained state.

The implant is generally dome-shaped adjacent a fold 104 in the braid resulting in a two-layer 106, 108 (inner and outer layer, respectively) construction The fold 104 in the braid is set at a tight radius, defining an aperture 110 closing the end of the implant.

Such features are more easily visualized in FIG. 2A showing one variation of the subject implant in cross section. In addition, one can see that the folded end of the implant 100 may be oriented proximally for use. As such, aperture 110 formed by the folded section (especially when held by a ring, band or tie 112) can be utilized as the interface for a detachable delivery system 150.

The opposite end of the implant may incorporate an inset hub or terminate with trimmed ends 114 (with or without incorporated polymer) or be otherwise configured as shown in FIG. 2B. The trimmed ends of braid may be coated with a polymer (e.g., TICOPHYLIC urethane by Lubrizol Advanced Materials, Inc.) to maintain a consolidated relationship of the wire ends.

Such coating is not a necessary provision. Instead, the implant is advantageously trimmed and subsequently handled to maintain braid integrity. Before describing these aspects, however, further reference is made to FIG. 2B.

Here, the implant 100′ is shown in association with a simple (i.e., non-detachable) pusher 150′. The pusher may be any elongate body ranging from a typical guidewire to a custom-made device. In any case, the pusher is used to track the device (possibly multiple devices as elaborated upon below) through a catheter/microcatheter (not shown) to the treatment site.

Features of interest incorporated in implant 100′ include a table or flat 114 across the side of the implant in which the doubled-up braid fold 104 is formed. Such an approach may assist in shaping the braid fold and also in driving implant shape recovery upon exit of the delivery catheter.

Another feature of interest is presented at the opposite side of the implant adjacent to where the free ends of the braid are trimmed. Namely, the braid is shaped with a conical inset 116. A simple conical shape (the triangular projection seen in cross-section) is advantageous. The free ends of the braid are urged inwardly relative to the implant body upon deployment by virtue of the crease/bend 118 formed in the braid. The depth, diameter and angle (α) of the inset, radius of the crease, and gap (G) between opposing ends of filaments in the braid of the conical inset can be varied. A tighter crease, higher cone angle and larger gap will help insure deployment of the feature.

In comparing FIGS. 2A and 2B, it is apparent that the folded-over (aperture 110) side of the implant can be oriented proximally or distally in relation to the delivery system for use. Even without taking advantage of the aperture for a detachment interface, it may still be desirable to orient the aperture proximally. This is the case because the bends forming aperture 104 may present a more stable face to the caging stent upon deployment. Even without a tie, the heatset of the braid maintains the aperture closure when the braid implant is deployed.

However, catheter tracking of the device may be improved if the rounded bend sections are oriented distally. Thus, depending on the circumstances, either orientation may be preferred.

Also, it is contemplated that more than one implant may be tracked through a delivery catheter/microcatheter at one time. The implants may be oriented in the same direction, or face opposite one another. In one advantageous configuration, two implants are loaded simultaneously, with the trimmed ends of each in contact. Such options are discussed further below in connection with FIGS. 5A-5D.

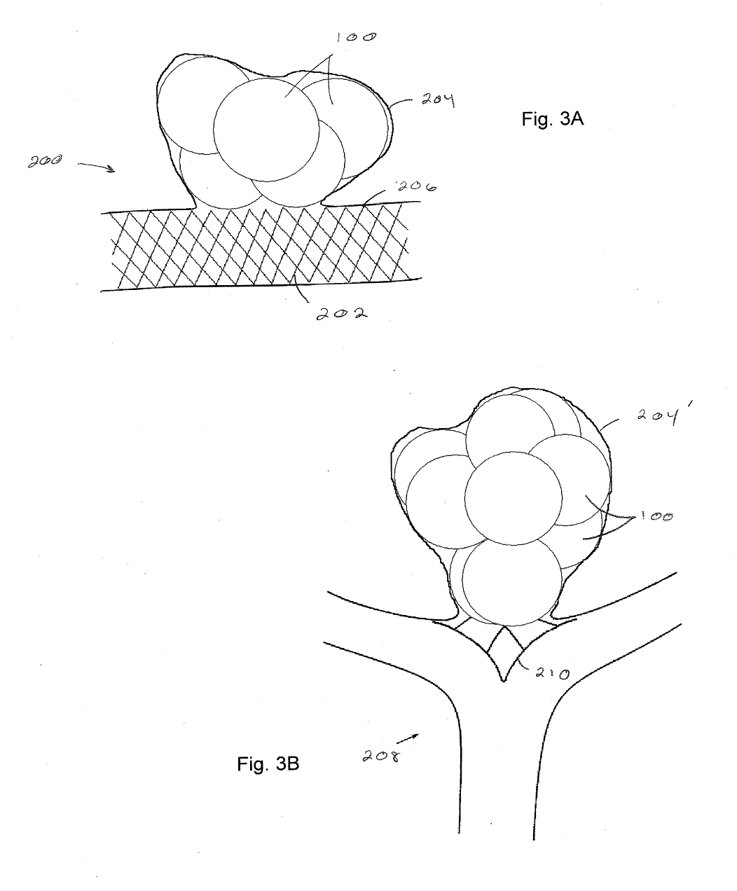

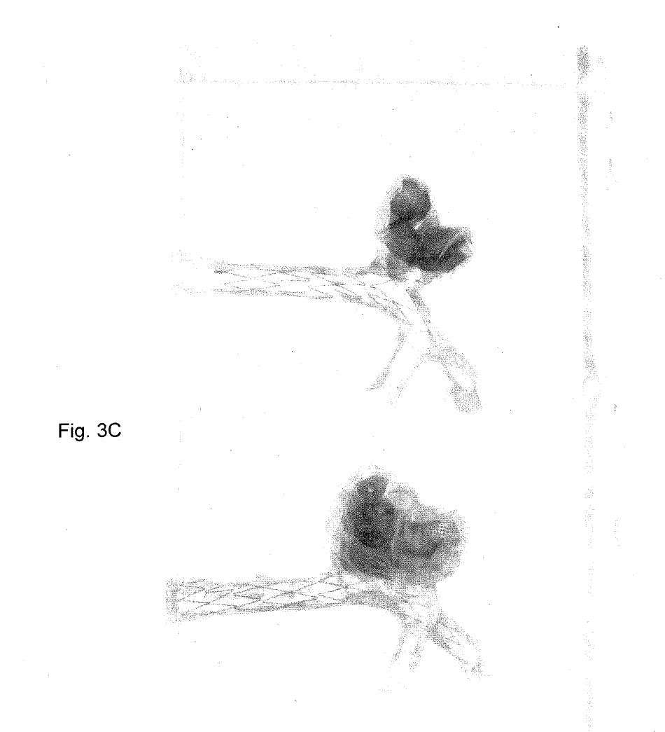

Irrespective of the delivery mechanics, FIGS. 3A-3D illustrate an intended result. In each figure, a complete treatment system 200 comprises multiple braid-ball implants 100 and a caging device. More specifically with respect to FIG. 3A, a “flow-disruptor” type stent 202 such as the PIPELINE (commercially available through Covidien, Inc.) is set across the neck of a side wall aneurysm 204. The ball-shaped implants 100 are delivered using a catheter “jailed” between the stent and the vessel wall 206 as per a technique common to aneurysm coiling. The result is multiple of the subject implants 100 sequestered within an otherwise challenging aneurysm shape.

Of course, a tube-cut stent such as the NEUROFORM or ENTERPRISE may be substituted for the PIPELINE stent—the former being employed in the model(s) pictured in FIG. 3C. An appreciation of the variety of aneurysms which the subject implants can be employed to treat is typically more important that selection of the stent itself. Because of their small size, in which a plurality of devices are used to at least partially fill an aneurysm, and the ability to retain them using any number of commercially available devices, the subject implants 100 are especially useful in treating irregularly shaped aneurysms (such as multi-lobular and fusiform types) not easily addressed with the other devices, either alone or in combination with coils, which often prolapse or otherwise protrude into the parent vessel in which a stent is placed.

The aneurysms appearing in FIGS. 3A and 3C are challenging because of irregular shape and large neck-to-dome length ratios. In addition, the placement of aneurysm 204′ in FIG. 3B at a vascular bifurcation 208 make is prone to recanalization (at least when coiling) due to the flow dynamics.

In treating a terminal aneurysm (such as the bifurcation aneurysm 208), a device like the now-defunct TRISPAN (commercially available through Target Therapeutics, Inc.) or neck bridge 210 (commercially available through Pulsar Medical Inc.) is placed across the neck of the aneurysm. Then, this device is “crossed” by a catheter to deliver the subject implants 100, which fill the available space, efficiently packing the aneurysm. Under fluoroscopy, the physician determines the number of implants to deliver in order to loosely or more densely pack the aneurysm. In either case, the braid matrix of each implant offers appreciable obstruction to flow and can quickly occlude the aneurysm as thrombus forms where flow is disrupted.

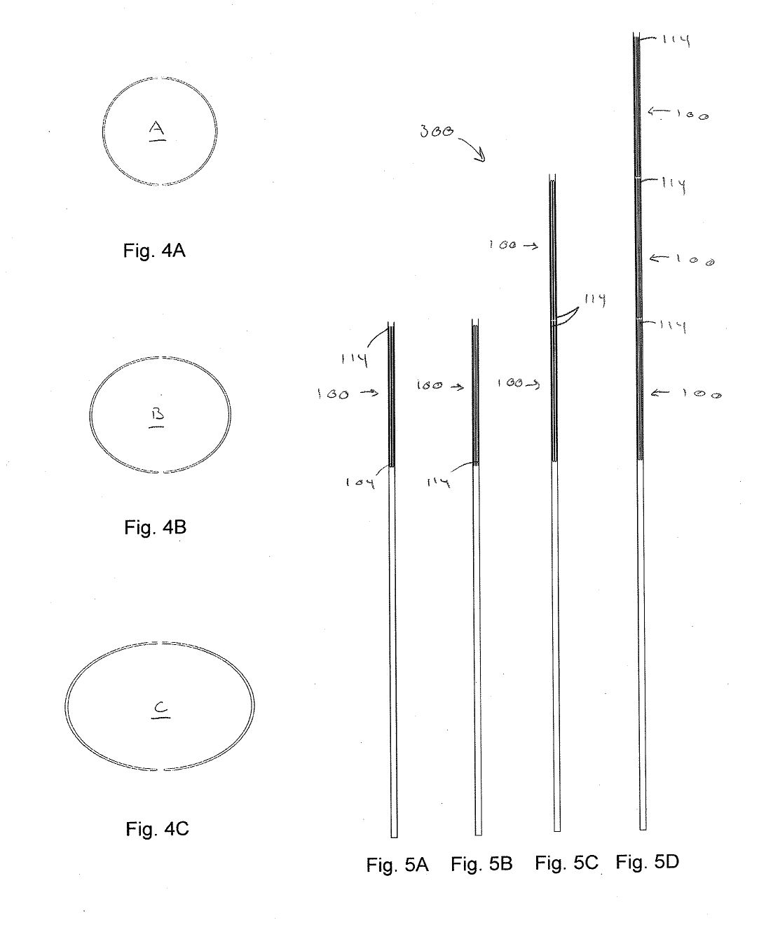

FIGS. 4A-4C illustrate the subject implant as provided in various relative (not actual) sizes A, B, C. The same or different-sized implants may be used in a given procedure or different sizes may be used for different treatment indications. For a selected braid configuration, it is notable that in larger sizes, the softness of the implant may call for maintaining the medial curvature of the device in order to drive full shape recovery upon deployment. Accordingly, although the implants increase is size/volume in examples A, B and C, they maintain approximately the same equatorial radius.

The subject implants may be loaded for use in a variety of ways. FIGS. 5A-5D illustrate implants in loading sheaths 300 (full-length or partial view) as typically employed with a variety of self-expanding interventional devices. The loading sheath may be constructed in a tear-away format such as produced by Galt, Inc. and may include handle features, perforations or other features not shown.

As referenced above, the implants 100 may be loaded into the sheaths according to different strategies. In FIG. 5A, the free ends 114 of the braid are oriented distally (and folded side 104 oriented proximally) for use. In FIG. 5B, the free ends 114 are set proximally. In FIG. 5C, two implants are loaded in the sheath together, with their free ends 114 in contact. In FIG. 5D three implants are provided, all facing in the same direction with their free ends 114 directed distally.

As noted above, for caging device interaction, it may advantageous to orient the free ends distally, leaving the more stable aperture section of the device to face proximally. As such, proximal-side contact between the subject implants 100 and the caging device is achieved minimizing the potential for free ends of the braid extending into blood flow past the caging device.

### Implant Manufacture

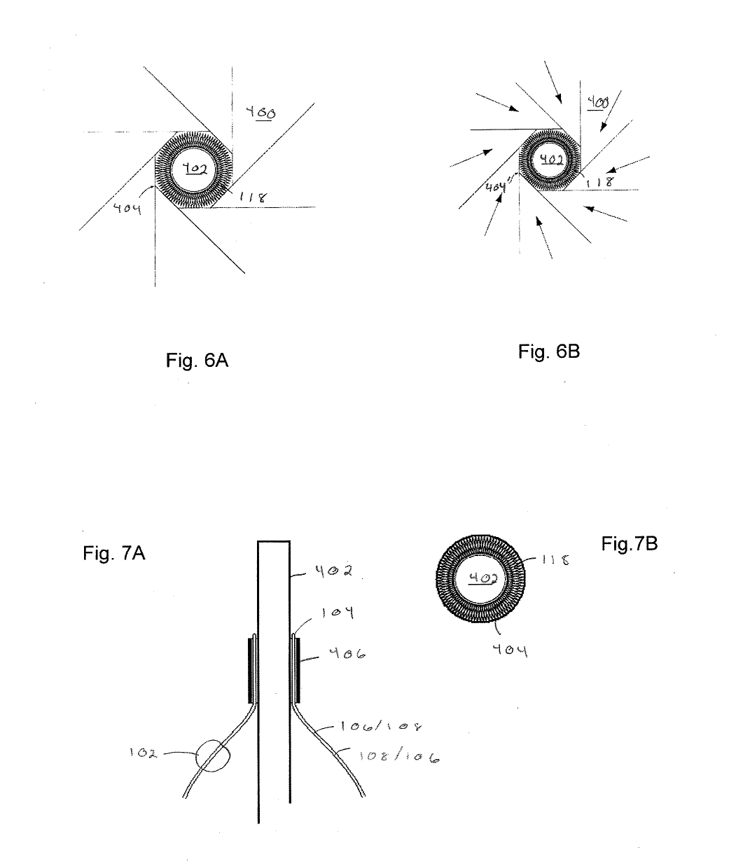

Prior to forming the gross/overall shape of the implant, the folded-over section may be heat treated into shape. Braid so-treated forms the stable, closed end of the device. FIGS. 6A and 7A show suitable tooling for forming braid into a minimally crimped bend into which it is heatset. In such a configuration, as illustrated in each of FIGS. 6B and 7B, the braid is essentially bottomed-out (or in the so-called “jam” condition) to that it is closed-off to the maximum extent possible (i.e., without buckling the braid matrix).

In the final implant construction, the braid may be tied closed in this position to define a fully immobile aperture for a delivery system interface (as shown in FIG. 2A) or simply retain stability in a closed position by virtue of the heatset imparted to the braid (as shown in the implant of FIG. 2B).

With specific reference to FIGS. 6A and 6B, these figures illustrate a crimper technique for presetting the shape of the implant fold. In FIG. 6A, wedges 400 of a crimper device (e.g., as available through Machine Solutions, Inc. and others) receive braid 102 that is folded over to define a plurality of individual filament bends 118. A mandrel 402 is advantageously set inside the braid. The mandrel limits compression of the braid tube, requiring the bends radius tighten when the cavity 404 formed by the wedges is closed as indicated in FIG. 6B. The shape of the fold is set by heat and/or a combination of strain and heat. The heat may be applied by a torch, within a furnace, by induction or—advantageously—by running current though the mandrel. In another approach, a multi-element chuck or collet type device is employed in a similar fashion to the crimper wedges illustrated above.

FIGS. 7A and 7B illustrate another pre-treatment approach for the fold. Specifically, the braid is pulled through a band or hypotube 406. Again, a mandrel 402 is set inside the braid to limit the inward bowing of the braid. Outward bowing is limited either by setting the braid fully within the band/hypotube or limiting its extension beyond the band. The braid 102 is then heat treated (e.g., as per above) either along its length or locally at the bend to set the tightly folded-over shape.

Note also, the fold/bend ultimately shown in FIG. 7B may be imparted in stages. For example, first a smaller mandrel may be used for a more relaxed fold with a first heat treat. Then, a tighter mandrel fit inside the braid to minimize the fold bend diameter. In another approach, the fold may first be heat treated some distance from the end of the band, then the band moved directly adjacent the bend to minimize it as shown in FIG. 7A for a secondary heat treatment.

In any case, the repeated heat treatment for the fold is not problematic given that oxides can be removed by etch and any changes to material properties has minimal effect because the closed end of the implant defined at the fold basically only pivots during delivery. In other words, the wire bends defining the closed end remain essentially stable during delivery and deployment, changing shape very little.

When pre-treating the braid, the fold may be formed by everting or inverting the braid. The result is that either layer 106/108 may be turned inside out in the final implant construction.

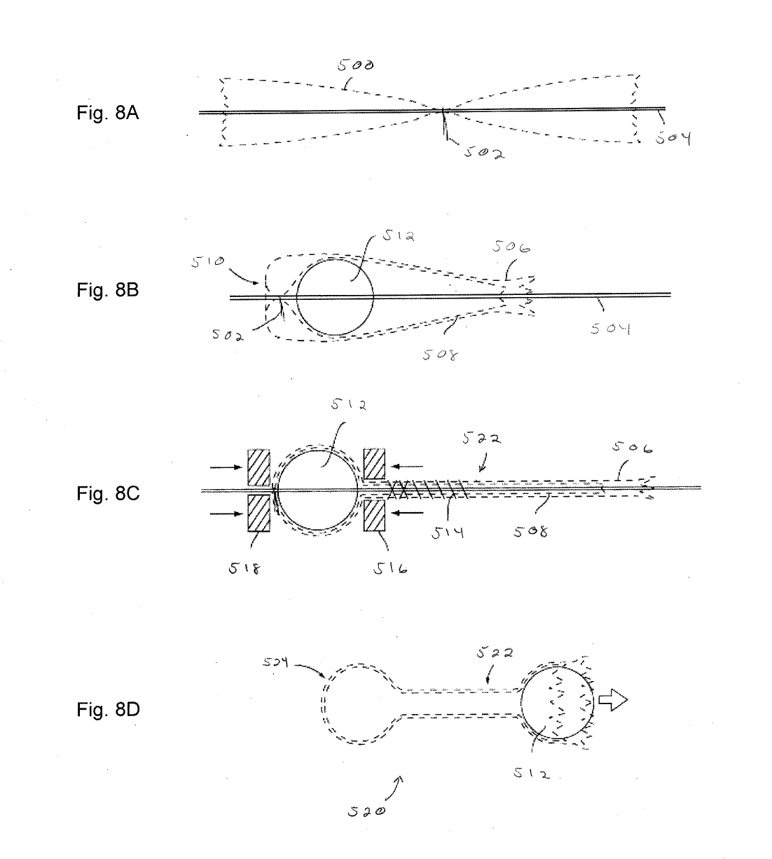

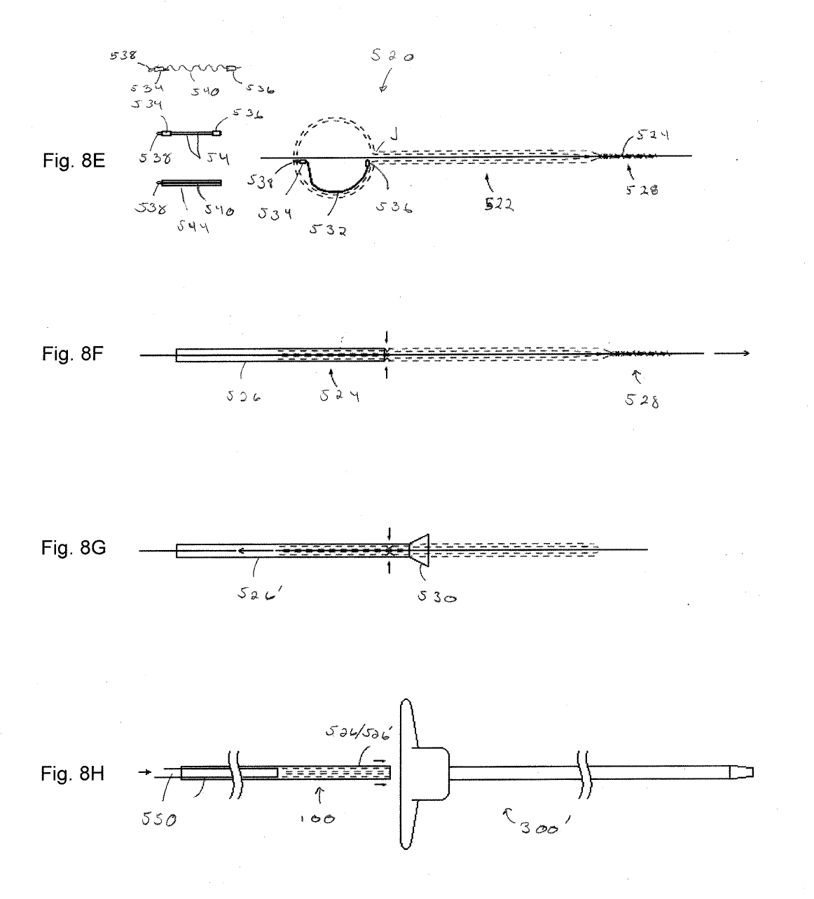

Regardless, with the braid so-shaped, the overall implant may be formed largely as described in connection with FIGS. 8C onward. This may occur with or without the use of the suture tie as described further below—instead relying on clamping pressure. Whatever approach employed, pre-treatment of the folded-over section can improve the consistency of the procedure described by handling the challenging aspect (i.e., bend/fold formation) of implant production in advance under highly constrained and controlled conditions.

Without reliance on pre-treating the bend, an optional manufacturing process begins with FIG. 8A. Here, a section of braid 500 is tied with suture 502 or a higher-strength filament/line alternative such as DYNEMA or SPECTRA—also referred to as “GSP” line (Gelspun Polyethylene)—upon a mandrel 504. The tie may be offset from where the braid is cut so when the braid is inverted as shown in FIG. 8B, that the outer layer 506 extends past the inner layer 508. A loose fold 510 is developed and the braid surrounds the implant shaping form 512.

In FIG. 8C, the braid is stretched and secured by wrap 514 (typically Pt, Nichrome or Stainless Steel wire) around the ball form 512. Compression forms 516, 518 are also shown (held by fixturing as indicated by arrows). Fold-side form 518 compresses the fold to a minimum profile during heat setting (e.g., for Nitinol braid at 500-550° C. for about 5 minutes).

At this stage, the braid is “folded-flat” within the meaning of the present invention. It may indeed be shaped across a flattened section of a ball as referenced above, or be substantially flat as formed at the apex of a sphere or ovaloid body. Even though it necessarily includes an aperture that may vary somewhat in size, is also “closed” as described above with respect to the pre-folded braid approaches discussed.

As per the approach in FIG. 8C, when heated, suture tie 502 burns away removing any impediment for achieving a zero or near-zero radius bend at the fold. Opposite form 516 optimally defines a sharp junction (to help define a clean indication or line for cutting when that end of the ball is to be trimmed, as described below) or an inset corresponding to a recess within form 516 to define a conical shape as pictured in FIG. 2B. Note that this junction (J) is indicated in FIG. 8E.

After shape-setting, a device perform 520 is ready once the internal tool piece is finally removed as illustrated in FIG. 8D. During this process, the ends of the braid are forced open and typically lose braid integrity/engagement. So that such action does not adversely affect the implant integrity, a “tail” 522 incorporated in the perform 520 should be sufficiently long (i.e., often about 2 cm or more) so as to avoid any damage from unraveled braid ends impacting the intended body 524 of the implant.

If the implant is formed from braid that includes an oxide layer, the perform is next etched, then passivated. However, if pre-etched wire is employed in braiding and any heatsetting performed in a salt pot, vacuum furnace, or using other equipment to minimize oxide formation, the perform may simply be subject to Nitric acid passivation. During any such etching, the length of the tail may likewise be useful for maintaining implant integrity through any implant manipulation that includes such as opening the end of the ball opposite the fold to ensure consistency.

After tying the outer layer 506 with a wrap 524 as shown in FIG. 8E, the tail of the implant is easily inserted (“back loaded”) into a working tube 526 for trimming as shown in FIG. 8F. Without the inner layer underneath, the tied section 528 offers an effective lead-in to the tube. Alternatively, the wrap (and any intentional difference in length between the inner and outer layers in the tail) may be omitted and the implant “front loaded” into a working tube 526′ including an introducer section 530 as shown in FIG. 8G.

Preferably set upon a mandrel (i.e., tied thereon as in FIG. 8E or inserted therein at FIG. 8G), the implant preform is trimmed to length (as indicated by the paired arrows) defining the final implant. The mandrel may be cut through with the implant or instead left intact to serve as a backing to help maintain braid integrity (e.g., if using a diamond wheel saw around the device instead of cutters).

Trimming at precisely the correct location is facilitated by the angular junction J that appears as a v-grove or notch when then the implant (body, tail or both) are constrained is a reduced diameter adjacent thereto. Form 516 optimally has no significant radius around the edge used to define the junction (other than a typical “break” to the edge as in common in machining practice). In any case, how “sharp” the angle that defines the junction (i.e., how small the radius at the junction) need be is driven by the associated functional utility in providing a clean cut line. Optionally, as an indication of where to cut, and also allowing for as small an associated aperture as desirable with the braid ends substantially tangent to the curvature of the implant body.

While generally to be avoided, a large aperture defined by the trimmed braid ends (ranging from about 1 to about 2 mm in diameter) or a “tail” remnant (ranging from about 0.5 to about 1 mm in length) may sometimes be acceptable. Yet, the former is non-optimal due to loss of implant size, matrix for flow disruption and tissue colonization and braid integrity. The latter is non-optimal given (at least the perception) of a potentially-traumatic nubbin. In any case, such a feature may be coated or potted in polymer (such as TICOPHYLIC) to either stabilize the braid and/or provide a “soft-tip” tissue interface.

Optional marker features as shown in FIG. 8E may also be incorporated in the device before trimming. A shaped tether 532 (e.g., made of NiTi ribbon) with proximal and distal markers (e.g., Pt bands) 534, 536 is set within the implant preform 520. Alternatively, it may be set between the braid layers. In either case, it may be secured by threading a loop 538 through one or more filaments of the braid located at the fold 510, or otherwise. The markers can be affixed by crimping, adhesive, etc. When the implant is constrained for delivery the preset shape on the tether straightens. The shape is resumed upon implant expansion in deployment.

Yet another “active” tether option is shown to the left of the implant preform. Here, tether 540 is set in a zig-zag or shallow helical configuration. When the implant is compressed, the tether is straightened and marker 536 located outside the interior of the braid-ball body. Upon deployment, the tether resumes its preset shape, pulling the marker into contact with the face of expanded implant (or potentially into the conical inset, if provided). Alternatively, the tether may pull marker 536 slightly into the interior of the expanded implant.

Instead of using a shaped tether, cost can be reduced by employing either one of alternate approaches shown to the left of the implant preform. In one approach, linear strand(s) 542 carry markers 534, 536 comprising more radiopaque material. The subassembly is sized to span the implant when in an expanded state. Using a more radiopaque material (such as platinum) for the strand(s) allows for an approach in which a polymer sleeve 544 is substituted for discrete marker bands. By wicking adhesive (e.g., LOCTITE 4014) into the sleeve or heating a heat-shrink sleeve to contract its diameter, the components are secured in a very cost-effective package.

Regardless of whether marker features are included, once trimmed the implant is ready for use. In which case, the working tube members 526/526′ may serve as the loading sheath 300 discussed above. However, as shown in FIG. 8H the implant may instead be transferred (e.g., using a push rod 550) into a typical loading sheath 300′.

The braid ends may undergo ultrasonic cleaning, passivation or other processing prior to loading for use and packaging. In any case, the bulk of the implant is intended to remain the working tube—ideally to maintain the relation of the free braid ends without disorganization—up to the point of implant transfer into the loading sheath or (using the working sheath as a loading sheath in a medical procedure) into a catheter hub for use.

Once the final implant 100 is loaded into the sheath 300′, the system is either then complete or one or more additional implants may be loaded into the sheath as described above. Moreover, the sheath may be sterile-packaged alone, or in combination with a pusher. Pairing the loaded sheath with a generic guidewire may offer consumers a particularly economically-advantageous bundle.

### Variations

The subject methods may include each of the physician activities associated with implant positioning and release. As such, methodology implicit to the positioning and deployment of an implant device forms part of the invention. Such methodology may include placing an implant within a brain aneurysm, or at parent vessel targeted for occlusion, or other applications. In some methods, the various acts of implant introduction to an aneurysm or parent vessel are considered. More particularly, a number of methods according to the present invention involve the manner in which the delivery system operates in reaching a treatment site, for example. Other methods concern the manner in which the system is prepared for delivering an implant.

Also, it is contemplated that any optional feature of the inventive variations described may be set forth and claimed independently, or in combination with any one or more of the features described herein. Reference to a singular item, includes the possibility that there is a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of the claim language. All references cited are incorporated by reference in their entirety. Although the foregoing invention has been described in detail for purposes of clarity of understanding, it is contemplated that certain modifications may be practiced within the scope of the appended claims.

1. An embolic device comprising braid forming inner and outer layers, the layers defining a shape adapted to compress for delivery through a catheter and expand upon release from constraint, the inner and outer layers meeting at a folded section, the improvement comprising:
the folded section set substantially flat when the device is uncompressed.

2. The device of claim 1, wherein the free ends of the braid opposite the folded section are trimmed substantially in-line with a contour of the device adjacent to the free ends.

3. The device of claim 1, wherein the free ends of the braid are on a first side of the device, and wherein the free ends of the braid are trimmed to define a conical inset section relative to a contour of the first side of the device.

4. The device of claim 1, further comprising proximal and distal radiopaque markers secured on an elongate member, wherein the elongate member is secured adjacent to the folded section, and the elongate member has a length substantially equal to a height of the device, with a proximal marker secured at or adjacent to a proximal end of the length.

5. An embolic device comprising:
braid forming inner and outer layers, the layers defining an at least substantially closed shape adapted to compress for delivery through a catheter and expand upon release from constraint,
the inner and outer layers meeting at a folded section defining a stable aperture at a first side of the device and with free ends of the braid at the other, second side of the device, and
wherein the free ends of the braid are trimmed substantially in line with a contour of the second side of the device.

6. A method of making an embolic device comprising:
producing a device preform including a body and a tail, with an angular junction between the body and the tail;
constraining the body adjacent to the junction;
trimming the tail at the junction; and
without allowing the body to open adjacent the junction, transferring the body into a tubular body.
